(19) United States
(12) Patent Application Publication
(10) Pub. No.: US 2021/0093289 A1
(43) Pub. Date: Apr. 1, 2021

(54) ULTRASOUND SYSTEM FOR IMPROVING NEEDLE VISUALIZATION

(71) Applicant: Cheng-Yuan HSIEH, Hsinchu City (TW)

(72) Inventor: Cheng-Yuan HSIEH, Hsinchu City (TW)

(21) Appl. No.: 17/024,742

(22) Filed: Sep. 18, 2020

Related U.S. Application Data
(60) Provisional application No. 62/906,159, filed on Sep. 26, 2019.

(30) Foreign Application Priority Data
Jun. 10, 2020 (TW) 109119554
Jun. 10, 2020 (TW) 109119555

(51) Int. Cl. A61B 8/08; G10K 11/30; A61B 8/00; G01S 15/89; G01S 15/58

(52) U.S. Cl. CPC A61B 8/0841; G10K 11/30; G01S 15/58; A61B 8/4488; G01S 15/8934; A61B 8/4494

(57) ABSTRACT

This invention provides an ultrasound system for improving needle visualization, including: an ultrasound transducer, which has a tail and a head. A plurality of transducing elements (or more specifically, piezoelectric elements) are embedded in a surface of the head. The transducing elements are arranged in an array of M multiplying N (M×N), wherein M is a positive even number, N is a positive integer, and N is greater than M.

### CROSS REFERENCE TO RELATED APPLICATION

This application claims the benefit of filing date of U.S. Provisional Application Ser. No. 62/906,159, entitled “Intelligent Stereotactic Ultrasound System for Needle Navigation” filed Sep. 26, 2019 under 35 USC § 119(e)(1).

This application claims the benefits of the Taiwan Patent Application Serial Number 109119554, filed on Jun. 10, 2020, the subject matter of which is incorporated herein by reference.

This application claims the benefits of the Taiwan Patent Application Serial Number 109119555, filed on Jun. 10, 2020, the subject matter of which is incorporated herein by reference.

### BACKGROUND OF THE INVENTION

### 1. Field of the Invention

This invention relates to an ultrasound system for improving needle visualization, in particular to an intelligent stereotactic ultrasonic system for needle navigation.

### 2. Description of Related Art

During a medical operation or a physical examination, a needle or needle-like instrument may be inserted into a patient's body. At this time, it must be ensured that the needle moves in the correct (moving) direction. For this purpose, an ultrasound transducer can be used to detect the position and direction of the needle.

FIGS. 1A and 1B show a prior art problem in needle navigation, typically called the “needle not in-plane” problem. A one-longitudinal-plane (“1L”, hereinafter) linear transducer 90 can detect the area of an image plane 92. However, as shown in FIG. 1A, in most cases, a needle 94 has only a small portion intersecting with the image plane 92, and the whole needle 94 does not appear on the image plane 92, so it is difficult to confirm the position and the direction of the needle 94.

Then, in order to adjust the needle 94 to let it become “in-plane”, a user must try to rotate the transducer 90 or reinsert the needle 94. However, if the user observes only the intersection of the needle 94 and the image plane 92 in FIG. 1A, the user may be confused for many possible directions of the needle, including the mirror image directions, as shown in FIG. 1B. Therefore, the user may try to find out the direction of the needle 94 by blindly rotating the transducer clockwise or anticlockwise, or reinserting the needle 94, but the user has a 50% possibility to rotate it to a wrong direction, and this is disadvantageous for an operation or a physical examination using the needle 94.

FIG. 1C shows a prior art solution for the aforementioned problem in needle navigation. An experienced user gets used to tilt the transducer, to incline the image plane, thereby collecting a plurality of inclined image planes, and they are helpful in determining the direction of the needle. Anyway, this kind of solution depends on the user's accumulated experience, and it provides extremely limited information about the direction of the needle, so it still has high probability for the user to rotate it to a wrong direction.

Therefore, it is desirable to provide an improved ultrasonic system, to mitigate or obviate the aforementioned problem.

### SUMMARY OF THE INVENTION

This invention provides a two-longitudinal-plane (“2L”, hereinafter) linear ultrasound transducer, applicable for navigating the orientation or the trajectory of a needle (or various needle-like instruments). The 2L linear ultrasound transducer and its various modifications as described afterwards are the realization of the invented “incomplete mixing technique of bilateral soundwaves”, and may be implanted to any “even-number”-longitudinal-plane linear transducer.

It should be noted that, in order to carry an intrinsic “central plane” in the midline of the transducer, a prior art multiple-longitudinal-plane linear ultrasound transducer carries “odd-number” columns of transducing elements and forms the same number of longitudinal planes, and a minimum number of three longitudinal planes (“3L”, hereinafter) are required. The importance of the central plane lies in that the user can identify the midpoint of the transducer as a mental imagery (or the corresponding position) of the central plane, and this ease of midpoint identification helps manual needle navigation. Furthermore, the prior art 3L (or odd-number-longitudinal-plane) ultrasound transducer may form a main central column, which improves image quality of a central plane (referring to FIGS. 9 and 10 afterwards). In view of this, the 2L (or even-number-longitudinal-plane) linear transducer of this invention not only intrinsically lacks a central plane, but also carries a central gap (referring to FIG. 5D). Therefore, various techniques are created to “rebuild” the central plane, or to minimize a blind zone under the central gap.

An “incomplete mixing technique of bilateral soundwaves” is invented and realized by various transducer design inventions mentioned afterwards, which enables the 2L linear transducer to rebuild its central plane. In particular, the “incomplete mixing” of the bilateral soundwaves means that the soundwaves from both the left and the right columns of transducing elements are partially mixed to a certain level while preserving their independencies or differences. Herein, the mixing of the bilateral soundwaves mitigates a “blind zone” caused by a gap (referring to FIG. 5D) between two columns of transducing elements, and makes the soundwaves possible to be transmitted to and received from the central plane to be built. Meanwhile, the preserved independencies of the bilateral soundwaves carry three dimensional information beyond the single longitudinal plane. Thus, the trajectory of a needle can be detected without mistaking its mirror image directions.

Comparing with the typical 1L linear transducer which faces problems of mirror images crossing its single longitudinal plane, the 2L linear transducer of this invention can confirm the trajectory of the needle by three dimensional soundwaves perception beyond the single longitudinal plane. While, comparing with the 3L linear transducer (wherein three is the minimum number for a prior art multiple-longitudinal-plane linear transducer), the 2L linear transducer of this invention has omitted a whole column of transducing elements, and therefore it can reduce the materials and the costs of a transducer.

According to one aspect of this invention, there is provided an ultrasound system for improving needle visualization, comprising an ultrasound transducer, which has a tail and a head; a plurality of transducing elements being embedded in a surface of the head, and the transducing elements are arranged in an array of M columns multiplying N rows, that is, M×N, wherein M is a positive even number, N is a positive integer, and N is greater than M. In a “non-folded” transducer, M may be 2, . . . , 8, 16, 24, or 32, for example.

Optionally, or preferably, the system further includes a central gap on the surface, the central gap dividing the array by column equally into a left column set of transducing elements and a right column set of transducing elements, configured to form a left ultrasonic longitudinal detection plane, namely a left plane, and a right ultrasonic longitudinal detection plane, namely a right plane, respectively; wherein there is a blind zone to be minimized under the central gap, or there is a bilateral-equal-time-distance central ultrasonic longitudinal detection plane, namely a central plane, to be built between the left plane and the right plane.

Optionally, or preferably, the central plane is reconstructed by algorithms analyzing time domain signal intensity from the left column set of transducing elements and the right column set of transducing elements, to intensify signal(s) converted from soundwave(s) from the central plane, or to cancel signal(s) converted from soundwave(s) from a position deviated from the central plane.

Optionally, or preferably, the left plane and the right plane are partially mixed based on an incomplete mixing technique of bilateral soundwaves, such that their soundwaves reach a certain level of mixing to build the central plane by signal processing, but at the same time, remain certain levels of their independencies to detect the trajectory of the needle.

Optionally, or preferably, to mix bilateral soundwaves, the surface of the head is folded to form a left regional surface embedded with the left column set of transducing elements, and a right regional surface embedded with the right column set of transducing elements, the left regional surface is not parallel to the right regional surface; or the surface of the head has a portion being a curved surface.

Optionally, or preferably, each column set of transducing elements itself has a bended structure with a convex surface or a concave surface, aiming at the blind zone or an object of interest.

Optionally, or preferably, to minimize the blind zone, an acoustic lens is attached in front of the transducing elements; the acoustic lens is configured such that the left plane intersects or partially overlaps the right plane, wherein the acoustic lens is a monofocal lens, or a multifocal lens which has a plurality of focuses; or the acoustic lens has one or more waveguide structures, aiming at the blind zone; wherein the focuses of the acoustic lens locate in line with the central gap, or locate in the central plane or the blind zone; or, the acoustic lens is in mirror symmetry with respect to the central plane.

Optionally, or preferably, to minimize the blind zone, a splitter is arranged between the transducing elements and the acoustic lens, or inside the acoustic lens, or in front of the acoustic lens, and the splitter aims at the blind zone; or, a splitting structure is formed in the acoustic lens, and the splitting structure aims at the blind zone.

Optionally, or preferably, the array is a staggered array, such that the left column set of transducing elements is misaligned from the right column set of transducing elements; or the array is a zipped array.

Optionally, or preferably, the system further includes an auxiliary signal source, attached on the ultrasound transducer or embedded inside the ultrasound transducer; the auxiliary signal source including one or more inertial measurement units, metal detectors, or magnetic sensors.

According to another aspect of this invention, there is provided an ultrasound system for improving needle visualization, comprising an ultrasound transducer, which has a tail and a head; wherein the head of the ultrasound transducer has surfaces embedded with a left 2D-array transducing elements module and a right 2D-array transducing elements module; wherein the modules are folded to each other, or not parallel to each other; or each module is composed of mutually misaligned micro transducing elements spreading on a convex, a concave, or a plane surface at the head, such that the modules aim at a blind zone or an object of interest.

Optionally, or preferably, to decrease the size of the central blind zone, the central gap is minimized, for example, to be less than 3 mm or even less than 2 mm; or a central column of transducing elements is inserted at the central gap, and extends the array of M×N into an extended array of (M+1)×N, wherein the extended array is divided into a left column set of transducing elements and a right column set of transducing elements by the central column of transducing elements, configured to form the left plane, and the right plane, and the central plane, respectively.

Optionally, or preferably, the central plane is built by algorithms analyzing the time domain signal intensity from the left column set of transducing elements, the central column of transducing elements, and the right column set of transducing elements, to intensify the signal(s) converted from the soundwave(s) from the central plane, or to cancel the signal(s) converted from the soundwave(s) from a position deviated from the central plane; thus, for a minimal array of 3×N when M equals two, a bilateral-augmented central plane is built.

Optionally, or preferably, to partially mix the bilateral soundwaves, an acoustic lens is attached in front of the transducing elements, such that the left plane intersects the right plane at the central plane. The structure, the function, and the effect of the acoustic lens for the (M+1)×N array of the transducing elements may refer to the acoustic lens for the M×N array of the transducing elements after suitable adjustment.

Optionally, or preferably, to partially mix the bilateral soundwaves, signal(s) from the left column set of transducing elements and signal(s) from the central column of transducing elements are mixed and processed by a left side circuit; the signal(s) from the central column of transducing elements and signal(s) from the right column set of transducing elements are mixed and processed by a right side circuit.

Optionally, or preferably, the left column set of transducing elements and the right column set of transducing elements have equal transverse dimension, and their transverse dimensions are equal to or wider than transverse dimension of the central column of transducing elements.

Optionally, or preferably, a left transducing element and a right transducing element have equal transverse dimension, and their transverse dimensions are equal to or wider than transverse dimension of a central transducing element.

Optionally, or preferably, the central column of transducing elements is not parallel to the left column set of transducing elements, and/or is not parallel to the right column set of transducing elements.

Optionally, or preferably, the central column of transducing elements is nearer to an object of interest than the left column set of transducing elements is, and/or than the right column set of transducing elements is; or the central column of transducing elements is farther away from an object of interest than the left column set of transducing elements is, and/or than the right column set of transducing elements is.

Optionally, or preferably, at least one of the transducing elements has a bended structure with a convex surface or a concave surface, aiming at a blind zone or an object of interest.

Other objects, advantages, and novel features of the invention will become more apparent from the following detailed description when taken in conjunction with the accompanying drawings.

### BRIEF DESCRIPTION OF THE DRAWINGS

FIGS. 1A and 1B show a prior art problem in needle navigation;

FIG. 1C shows a prior art solution for the aforementioned problem in needle navigation;

FIG. 2 shows a perspective drawing of the two-longitudinal-plane (2L) linear ultrasound transducer according to one embodiment of this invention;

FIGS. 3A, 3B, and 3C show schematic diagrams illustrating the operation of the ultrasound transducer in FIG. 2;

FIGS. 4A, 4B, and 4C show the implementations of the “incomplete mixing technique of bilateral soundwaves” to the two-longitudinal-plane (2L) linear ultrasound transducers according to several embodiments of this invention;

FIG. 5A shows a schematic diagram of the structure of the acoustic lens;

FIG. 5B show the combinations of the acoustic lenses according to several embodiments of this invention;

FIG. 5C shows the acoustic lens having one or more waveguide structures according to one embodiment of this invention;

FIG. 5D shows a schematic diagram about the operation of the acoustic lens;

FIG. 6A shows a schematic diagram of the two-longitudinal-plane linear ultrasound transducer having the folded structure according to one embodiment (called “2L linear, folded”) of this invention;

FIG. 6B shows a schematic diagram of the two-longitudinal-plane linear ultrasound transducer having the folded structure and working with the acoustic lens according to one embodiment (called “2L linear, folded”) of this invention;

FIGS. 6C, 6D, and 6E show the configurations of the ultrasound transducers according to several embodiments of this invention;

FIGS. 6F, 6G, and 6H show the configurations of the ultrasound transducers having convex transducing elements and working with the acoustic lens according to several embodiments of this invention;

FIG. 7A shows the ultrasound transducer having the staggered array according to one embodiment of this invention;

FIG. 7B shows the size (or the dimension) of the transducing element;

FIG. 7C shows the two-longitudinal-plane linear ultrasound transducer having the folded structure and the staggered array according to one embodiment (called “2L linear, staggered, folded”) of this invention;

FIG. 7D shows the ultrasound transducer capable of detecting an image from a transverse plane and having the staggered array according to one embodiment of this invention;

FIGS. 7E, 7F, and 7G show the ultrasound transducers having the zipped arrays according to several embodiments of this invention;

FIG. 8 shows a block diagram of the ultrasound system for improving needle visualization according to one embodiment of this invention;

FIG. 9 shows a perspective drawing of the three-longitudinal-plane (3L) linear ultrasound transducer having a main central column according to one comparative example of this invention;

FIG. 10 shows a perspective drawing of the three-longitudinal-plane linear ultrasound transducer having an ancillary central column according to one embodiment (called “3L linear, ancillary central”) of this invention;

FIG. 11A shows a functional circuit block diagram of the three-longitudinal-plane (3L) ultrasound transducer according to one embodiment of this invention;

FIG. 11B shows a functional circuit block diagram of the five-longitudinal-plane (5L) ultrasound transducer according to another embodiment of this invention;

FIG. 12 shows a functional circuit block diagram of the ultrasound transducer utilizing the 2D-array transducing element module according to one embodiment of this invention;

FIGS. 13A and 13B show the configurations of the three-longitudinal-plane (3L) ultrasound transducers according to several embodiments of this invention;

FIGS. 14A, 14B, 14C, and 14D show the configurations of the ultrasound transducers utilizing the 2D-array transducing element modules according to several embodiments of this invention;

FIGS. 15A and 15B respectively show side views, first perspective drawings, and second perspective drawings of the 2L transducer and the 3L transducer each having curved surfaces.

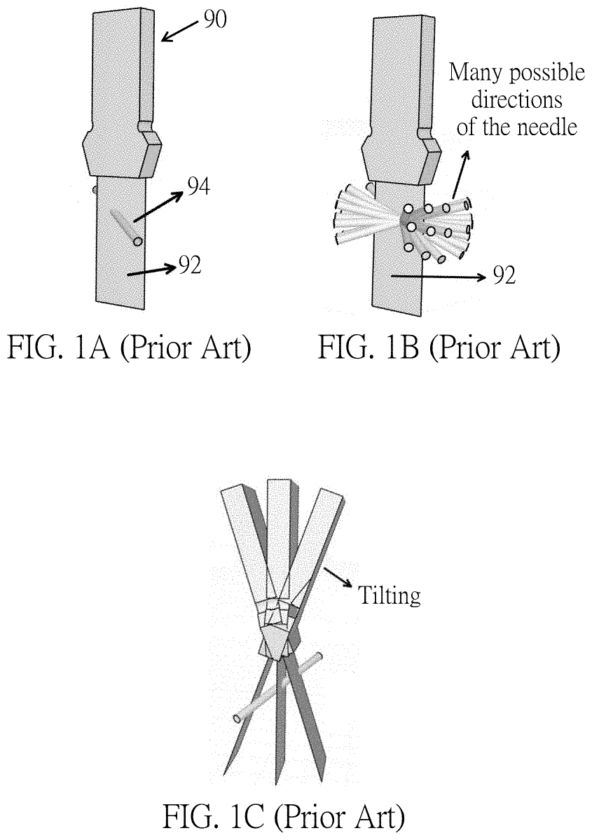

### DETAILED DESCRIPTION OF THE EMBODIMENT

Different embodiments of the present invention are provided in the following description. These embodiments are meant to explain the technical content of the present invention, but not meant to limit the scope of the present invention. A feature described in an embodiment may be applied to other embodiments by suitable modification, substitution, combination, or separation.

It should be noted that, in the present specification, when a component is described to have an element, it means that the component may have one or more of the elements, and it does not mean that the component has only one of the element, except otherwise specified.

Moreover, in the present specification, the ordinal numbers, such as “first” or “second”, are used to distinguish a plurality of elements having the same name, and it does not means that there is essentially a level, a rank, an executing order, or an manufacturing order among the elements, except otherwise specified. A “first” element and a “second” element may exist together in the same component, or alternatively, they may exist in different components, respectively. The existence of an element described by a greater ordinal number does not essentially means the existent of another element described by a smaller ordinal number.

Moreover, in the present specification, a feature A “or” or “and/or” a feature B means that A exists individually, B exists individually, or A and B exist together; a feature A “and” a feature B means that A and B exist together; the terms “include”, “comprise”, “have”, or “contain” means “include but not limited thereto”, except otherwise specified.

Moreover, in the present specification, the terms, such as “top”, “bottom”, “left”, “right”, “front”, “back”, or “middle”, as well as the terms, such as “on”, “above”, “under”, “below”, or “between”, are used to describe the relative positions among a plurality of elements, and the described relative positions may be interpreted to include their translation, rotation, or reflection.

Moreover, in the present specification, when an element is described to be arranged “on” another element, it does not essentially means that the elements contact the other element, except otherwise specified. Such interpretation is applied to other cases similar to the case of “on”.

Moreover, in the present specification, the terms, such as “preferably” or “advantageously”, are used to describe an optional or additional element or feature, and in other words, the element or the feature is not an essential element, and may be ignored in some embodiments.

Moreover, in the present specification, when an element is described to be “suitable for” or “adapted to” another element, the other element is an example or a reference helpful in imagination of properties or applications of the element, and the other element is not to be considered to form a part of a claimed subject matter; similarly, except otherwise specified; similarly, in the present specification, when an element is described to be “suitable for” or “adapted to” a configuration or an action, the description is made to focus on properties or applications of the element, and it does not essentially mean that the configuration has been set or the action has been performed, except otherwise specified.

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

Moreover, in the present specification, the terms, such as “system”, “apparatus”, “device”, “module”, or “unit”, refer to an electronic element, or a digital circuit, an analogous circuit, or other general circuit, composed of a plurality of electronic elements, and there is not essentially a level or a rank among the aforementioned terms, except otherwise specified.

Moreover, in the present specification, two elements may be electrically connected to each other directly or indirectly, except otherwise specified. In an indirect connection, one or more elements, such as resistors, capacitors, or inductors may exist between the two elements. The electrical connection is used to send one or more signals, such as DC or AC currents or voltages, depending on practical applications.

[Two-Longitudinal-Plane (2L) Linear Ultrasound Transducer]

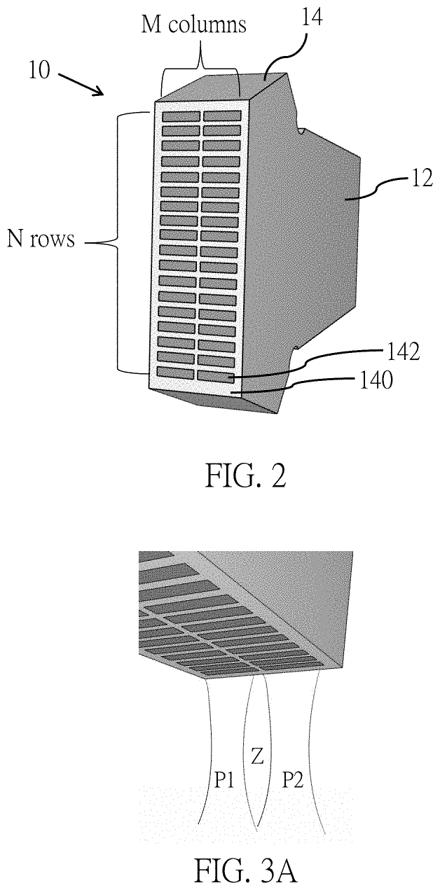

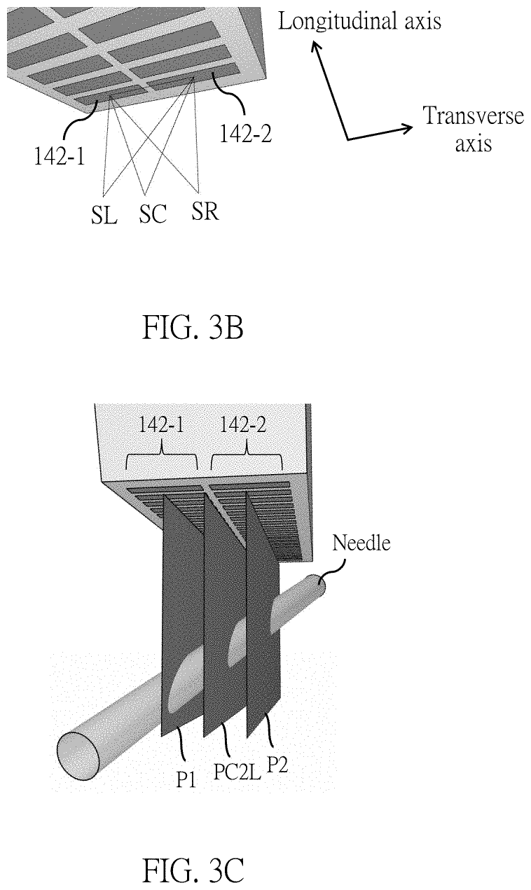

FIG. 2 shows a perspective drawing of the two-longitudinal-plane (2L) linear ultrasound transducer 10 according to one embodiment of this invention. FIGS. 3A, 3B, and 3C show schematic diagrams illustrating the operation of the ultrasound transducer 10 in FIG. 2.

The ultrasound transducer 10 of this invention has a tail 12 and a head 14. The tail 12 is held by a user (or a manipulator, etc.). A plurality of (electroacoustic) transducing elements 142 are embedded in a surface 140 of the head 14. In other words, the transducing elements 142 typically do not protrude from the surface 140. During operation, the surface 140 can be closely abutted to a body part under detection, such as a patient's surgical site. In other embodiments, other components, such as an acoustic lens or an acoustic matching layer, may be further arranged on the surface 140, and in those cases, what is closely abutted to the body part under detection may be those components rather than the surface 140. The transducing element 142 may be a piezoelectric element. The transducing element 142 can convert ultrasonic wave(s) into electronic signal(s), or convert electronic signal(s) into ultrasonic wave(s), thereby receiving or transmitting the ultrasonic wave(s). (Ultrasonic wave is a type of soundwave.) According to this invention, the transducing elements 142 of the ultrasound transducer 10 are arranged in an array of M columns multiplying N rows (M×N), wherein M is a positive even number, N is a positive integer, and N is greater than M. In a “non-folded” transducer, M may be 2, . . . , 8, 16, 24, or 32, for example.

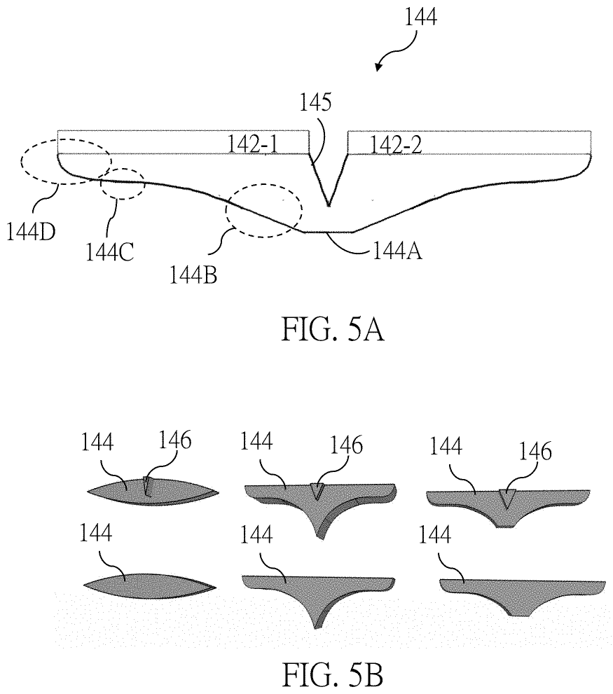

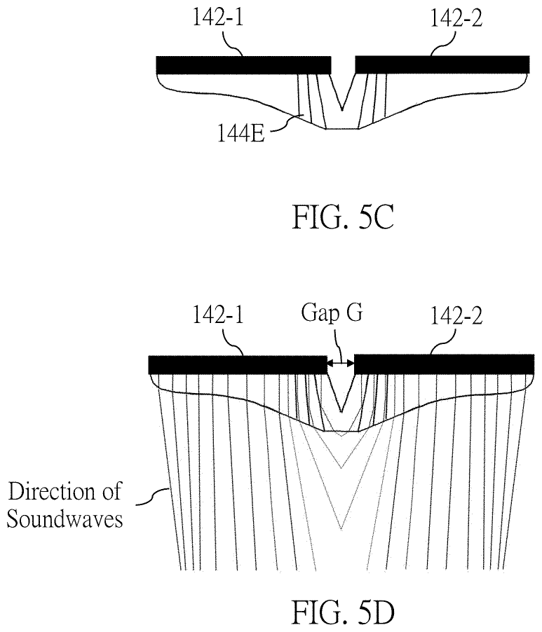

As shown in FIG. 2, when M=2, the aforementioned array is an array having two columns and N rows, and a central gap G (referring to FIG. 5D) divides the transducing elements 142 into a first column set 142-1 of transducing elements and a second column set 142-2 of transducing elements, configured to form a first ultrasonic longitudinal detection plane P1 (the “first plane” or the “left plane” in the basic embodiment of the present invention, hereinafter) and a second ultrasonic longitudinal detection plane P2 (the “second plane” or the “right plane” in the basic embodiment of the present invention, hereinafter), respectively. The longitudinal detection plane is called “longitudinal plane” hereinafter.

As shown in FIG. 3A, when M=2, between the first plane P1 and the second plane P2, there is a bilateral-equal-time-distance central ultrasonic longitudinal detection plane PC2L to be built (referring to FIG. 3C, also called a “central plane”, hereinafter), or a blind zone Z to be mitigated or minimized.

Therefore, this invention introduces various techniques and algorithms to rebuild the central plane PC2L, or compensate for the vacancy of the blind zone Z. As assumed in FIG. 3B, three points of echo signals SL, SC, and SR locate on the first plane P1, the central plane PC2L, and the second plane P2, respectively. The ultrasound system algorithms of this invention can rebuild an additional central image plane by performing time domain analysis on bilateral echo signals. That is, the algorithm intensifies only the signals SC from the central plane by its equal time distance to the first column set of transducing elements 142-1 and the second column set of transducing elements 142-2. Meanwhile, the algorithms cancel only the signals SR and SL deviated from the central plane by their different time distances to the corresponding transducing elements 142-1 and 142-2.

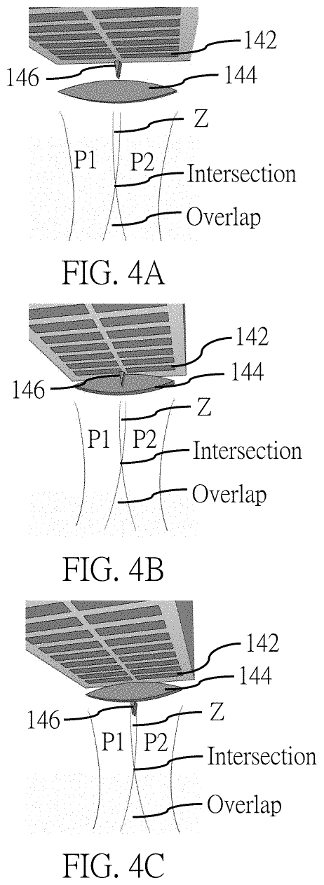

FIGS. 4A, 4B, and 4C show the implementations of the “incomplete mixing technique of bilateral soundwaves” to the two-longitudinal-plane (2L) linear ultrasound transducers according to several embodiments of this invention.

As shown in FIGS. 4A, 4B, and 4C, an acoustic lens 144 may be attached in front of the transducing elements 142. The acoustic lens 144 is configured to encompass different columns of transducing elements, such that the first plane P1 intersects or partially overlaps the second plane P2.

To further minimize the blind zone Z, a splitter 146 may be arranged between the transducing elements 142 and the acoustic lens 144 (as shown in FIG. 4A), or inside the acoustic lens 144 (as shown in FIG. 4B), or in front of the acoustic lens 144 (as shown in FIG. 4C), and the splitter 146 aims at the blind zone Z or the bilateral-equal-time-distance central ultrasonic longitudinal detection plane PC2L, thereby guiding the soundwaves to the blind zone Z, or guiding the echoes from the blind zone Z to the transducing elements 142.

In this way, soundwaves from both of the left side and the right side, i.e. the soundwaves from the first plane P1 and the second plane P2 are “incompletely mixed”. This “incomplete mixing” enables a 2L linear transducer to function as a 3L linear transducer. The mixing of the bilateral soundwaves can mitigate a blind zone caused by a gap G (as shown in FIG. 5D) between two columns of transducing elements 142, and make targets in the central plane PC detectable. On the other hand, the independencies of the bilateral soundwaves allow the transducer to carry three dimensional perception beyond the single longitudinal plane, so as to confirm the direction of the needle, and avoid mistaking its mirror image directions.

A conventional multiple-longitudinal-plane (equal to or more than three planes) transducer works by focusing and imaging independently among different longitudinal planes. To parallelly delineate the orientable space, the different longitudinal planes do neither intersect nor overlap each other in the space. To minimize soundwaves traveling across neighboring columns, isolated acoustic lenses may be mounted at each column of transducing elements for independent focusing within the corresponding column.

However, in this invention, the incomplete mixing of the bilateral soundwaves is the key innovation and is achieved by one or more of the following means: (i) an acoustic lens for encompassing multiple columns, an acoustic lens waveguide, an acoustic lens splitter; (ii) arranging the transducing elements in a transducer having a folded structure; (iii) arranging the transducing elements in a zipped array; and so on.

In terms of advantages, comparing with the typical 1L linear transducer, the 2L ultrasound transducer 10 of this invention can provide three dimensional perception beyond the single longitudinal plane to confirm the direction of the needle, and avoid mistaking the mirror image directions. While, comparing with the 3L linear transducer, the ultrasound transducer 10 of this invention has omitted a whole (central) column of transducing elements, and therefore it can reduce the complexity and the costs of the transducer.

FIG. 5A shows a schematic diagram of the structure of the acoustic lens 144. FIG. 5B shows the combinations of the acoustic lenses according to several embodiments of this invention. FIG. 5C shows the acoustic lens having one or more waveguide structures 144E according to one embodiment of this invention. FIG. 5D shows a schematic diagram about the operation of the acoustic lens. Herein, only one transducing element in one row of the first column set 142-1 of transducing elements and only one transducing element in one row of the second column set 142-2 of transducing elements are schematically labeled.

The acoustic lens 144 of this invention is used to refract soundwaves from bilateral columns of transducing elements for minimizing the blind zone Z under the central gap G and for central plane reconstruction. The acoustic lens 144 is designed to encompass multiple columns of transducing elements, rather than a “one lens by one column” design seen in a traditional multiple-longitudinal-plane transducer. The acoustic lens 144 may include the following components in an order from the center to the left as shown in FIG. 5A: (i) a central blunt cap 144A, which can reduce the wear of the central portion of the acoustic lens 144; (ii) a splitting structure 145 aiming at the blind zone Z, wherein the splitting structure 145 may be hollow (and filled with air), or may be filled with a splitter 146 as shown in FIG. 5B, and the material of the splitter 146 is different from the material of the acoustic lens 144 to provide different acoustic refractive indices; (iii) a concave lens structure 144B; (iv) an inflection point 144C; and (v) a convex lens structure 144D. The aforementioned components (i) to (v) may be integrated to form a multifocal lens, which has multiple focuses on the bilateral-equal-time-distance central plane PC2L or the plane containing the blind zone Z below the gap G. Accordingly, the effective lens encompasses bilateral columns of transducing elements, and the lens is geometrically symmetric with respect to the bilateral-equal-time-distance central plane PC2L, so the acoustic lens 144's structure from the center to the right can refer to its structure from the center to the left.

In the embodiments, the so-called concave lens, inflection point, or convex lens, are defined in view of the center of a transducing element in a single side or the detection planes PL and PR, and their local characteristics may vary following different acoustic media with different acoustic refractive indices.

The three embodiments as shown in the upper side of FIG. 5B use the acoustic lens 144 and the splitter 146 formed separately, while, the three embodiments as shown in the lower side of FIG. 5B only use the acoustic lens 144, but do not use any splitter 146.

Furthermore, as shown in FIG. 5C, the acoustic lens 144 may also have one or more waveguide structures 144E, the soundwaves passing through the acoustic lens 144 having the waveguide structures 144E have direction as shown in FIG. 5D, and it is noted that the blind zone Z in FIG. 3B has been minimized or even disappeared.

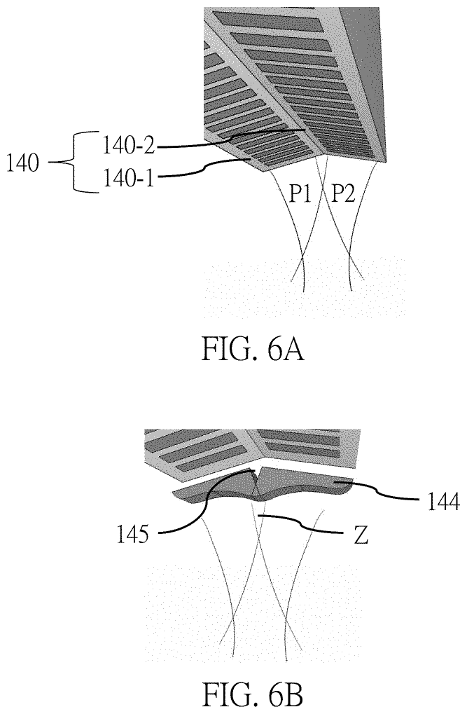

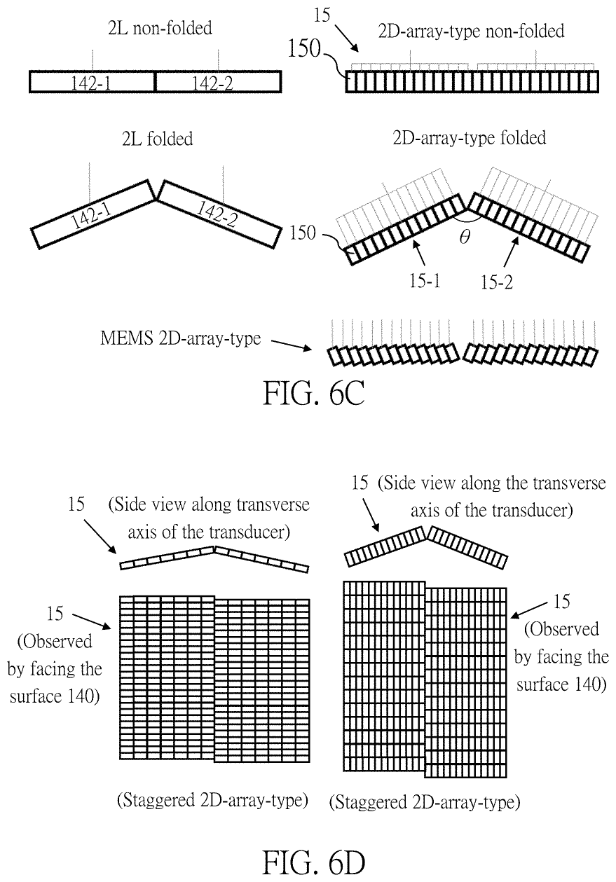

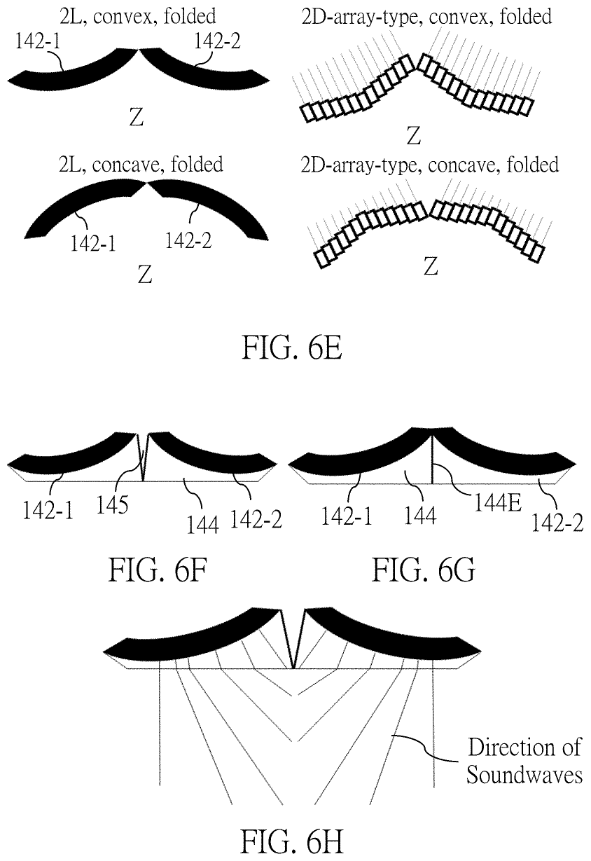

FIG. 6A shows a schematic diagram of the two-longitudinal-plane linear ultrasound transducer having the folded structure according to one embodiment (called “2L linear, folded”) of this invention. FIG. 6B shows a schematic diagram of the two-longitudinal-plane linear ultrasound transducer having the folded structure and working with the acoustic lens 144 according to one embodiment (called “2L linear, folded”) of this invention.

As shown in FIG. 6A, the surface 140 is folded to form a first regional surface 140-1 embedded with a first column set 142-1 of transducing elements, and a second regional surface 140-2 embedded with a second column set 142-2 of transducing elements. The first regional surface 140-1 is not parallel to the second regional surface 140-2. In particular, the angle between them is chosen such that the first plane P1 and the second plane P2 intersect or partially overlap (within a specific depth).

Although in the case of FIG. 6A, the first plane P1 has already intersected or partially overlapped the second plane P2, it is still possible to introduce the acoustic lens 144 to enhance the mixing of the soundwaves from both the left side and the right side, as shown in FIG. 6B. In the case of FIG. 6B, the acoustic lens 144 is a multifocal lens formed with a splitting structure 145 aiming at the blind zone Z.

FIGS. 6C, 6D, and 6E show the configurations of the ultrasound transducers according to several embodiments of this invention. Herein, only one transducing element in one row of the first column set 142-1 of transducing elements and only one transducing element in one row of the second column set 142-2 of transducing elements are schematically labeled.

The upper left diagram denoted as “2L non-folded” in FIG. 6C is the simplified schematic side view of the embodiment in FIG. 2. The middle left diagram denoted as “2L folded” in FIG. 6C is the simplified schematic side view of the embodiment in FIG. 6A.

Comparably, in the upper right diagram of FIG. 6C denoted as “2D-array-type non-folded”, the transducing elements 142 are replaced by a 2D-array-type transducing element module 15. The 2D-array-type transducing element module 15 intrinsically has a plurality of micro transducing elements 150, and they are divided into a plurality of groups. One group of micro transducing elements 150 may be used as a single transducing element 142.

Comparably, in the middle right diagram of FIG. 6C denoted as “2D-array-type folded”, the 2D-array-type transducing element module 15 are divided into a first group 15-1 (which replaces the first column set 142-1 of transducing elements) and a second group 15-2 (which replaces the second column set 142-2 of transducing elements) of micro transducing elements 150, and the two groups are folded to have an angle θ between them from 90 degrees to 180 degrees, so they may be parallel or not parallel to each other.

Comparably, in the lower right diagram of FIG. 6C denoted as “MEMS 2D-array-type”, a microelectromechanical system (MEMS) forms the micro transducing elements 150 of the 2D-array-type transducing element module 15. In this case, although the micro transducing elements are arranged in a line, all of them aim at the central plane PC2L (referring to FIG. 3C).

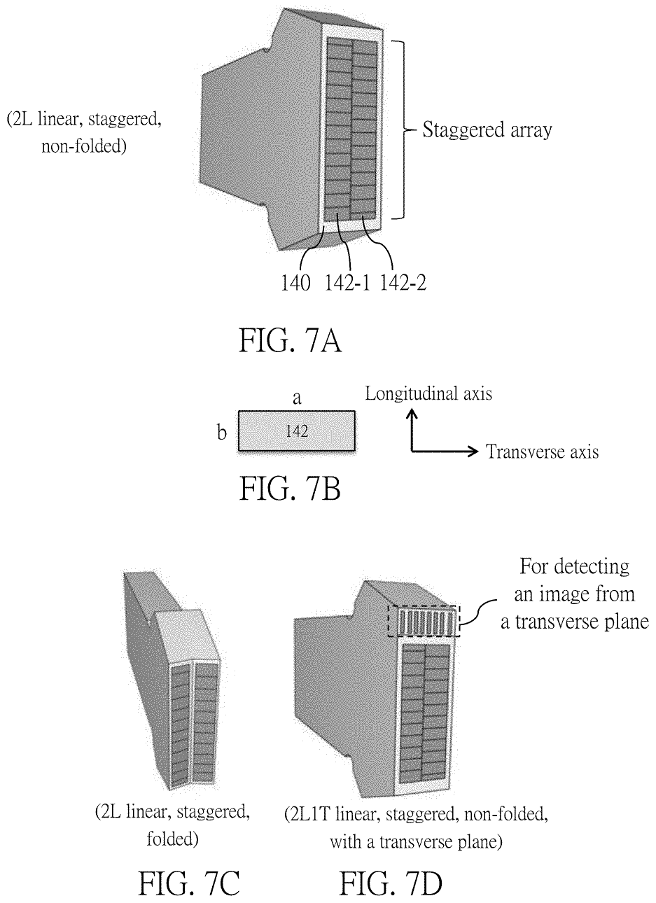

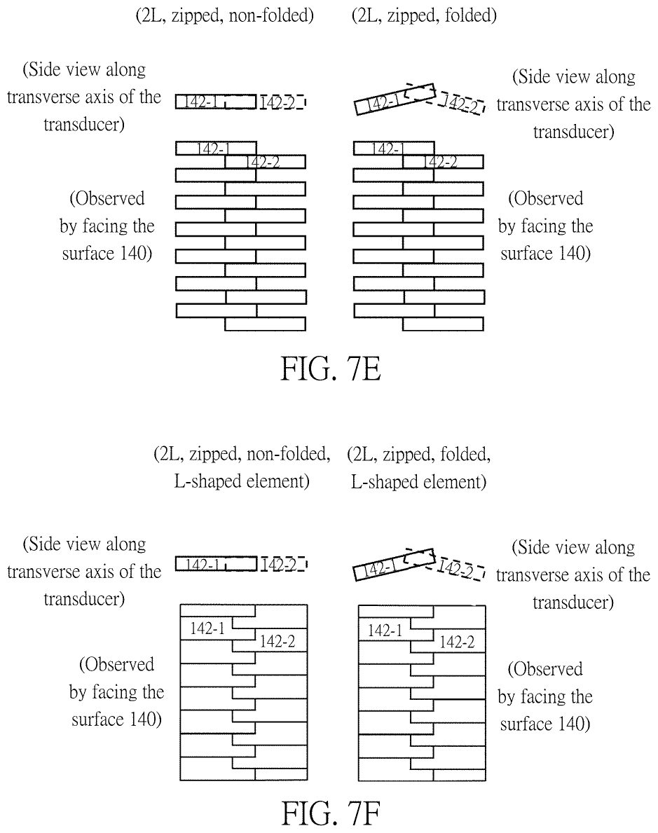

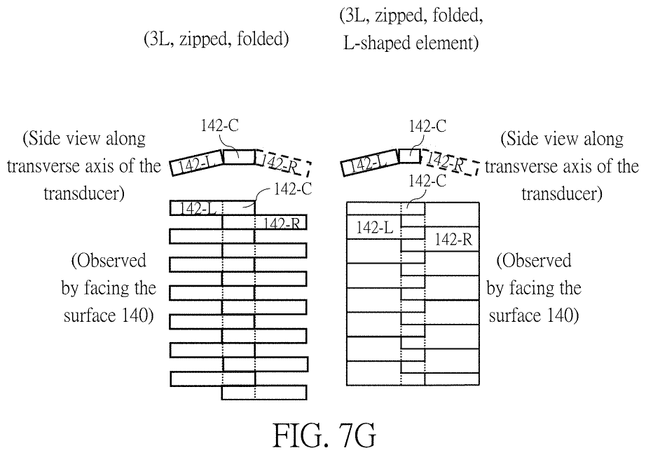

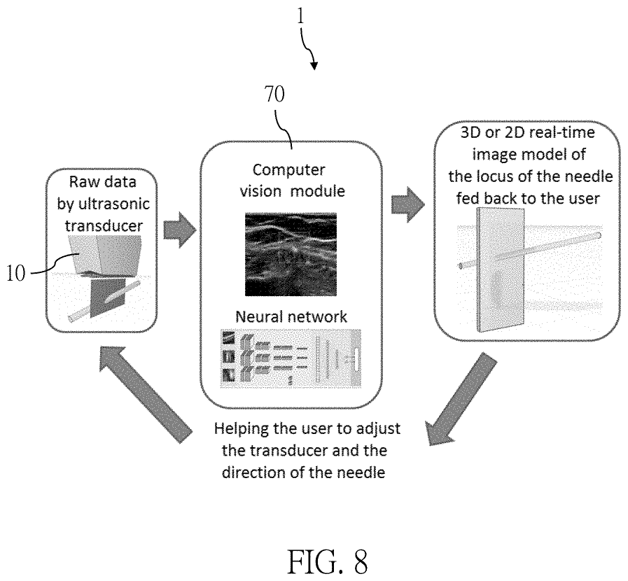

FIG. 6D shows schematic diagrams of two types of “staggered 2D-array-type” transducing element modules 15 from different view angles. The two types of 2D-array-type transducing element modules 15 are different in that the length and the width of the micro transducing elements 150 are exchanged. The function of the “staggered” arrangement may refer to descriptions in FIG. 7A.

In FIG. 6E, each set 142-1 and 142-2 of transducing elements itself has a bended structure with a convex surface or a concave surface, aiming at the blind zone Z or the object of interest. In two alternative cases here formed by 2D-array-type transducing element modules 15, the micro transducing elements 150 may also be misaligned to each other, such that each group 15-1 and 15-2 of the 2D-array-type transducing element module 15 has a bended structure with a convex surface or a concave surface, aiming at the blind zone Z or the object of interest.

FIGS. 6F, 6G, and 6H show the configurations of the ultrasound transducers having convex transducing elements 142-1 and 142-2 and working with the acoustic lens 144 according to several embodiments of this invention, wherein the acoustic lens 144 may have a splitting structure 145 or waveguide structure(s) 144E, and its specific structure may refer to FIG. 5A and its relevant descriptions, so the details are omitted here.

FIG. 7A shows the ultrasound transducer having the staggered array according to one embodiment of this invention, wherein the first column set 142-1 of transducing elements is misaligned or staggered from the second column set 142-2 of transducing elements. FIG. 7B shows the size (or the dimension) of the transducing element 142, wherein the transducing element 142 has a length of “a” unit and a width of “b” unit. In the staggered array in FIG. 7A, the misalignment between the two column of transducing elements 142 can yield higher resolution for the central plane PC. Specifically, assuming that an ultrasound transducer without a misalignment configuration has a resolution of “R” ppi (pixels per inch), then, an ultrasound transducer with a misalignment of “0.5b” unit can have an improved resolution between “R” ppi to “2R” ppi.

FIG. 7C shows the two-longitudinal-plane linear ultrasound transducer having the folded structure and the staggered array according to one embodiment (called “2L linear, staggered, folded”) of this invention. It functions as previously discussed, so the details are omitted here.

FIG. 7D shows a “2L1T linear, staggered, non-folded” ultrasound transducer capable of detecting a transverse image plane according to one embodiment of this invention, wherein the direction of the transducing elements 142 used to detect the transverse image plane is perpendicular to the direction of the transducing elements 142 used to detect the longitudinal image plane. The transverse image plane collaborates with two longitudinal image planes to detect the trajectory of the needle more precisely.

FIGS. 7E, 7F, and 7G show the ultrasound transducers having zipped arrays according to several embodiments of this invention. FIGS. 7E and 7F both show the 2L linear transducers, and FIG. 7G shows the 3L linear transducer. Herein, the side view along the transverse axis of the transducer only illustrates one transducing element in one row of each column set 142-1 and 142-2 of transducing elements. Furthermore, the first column set 142-1 of transducing elements marked by the solid lines and the second column set 142-2 of transducing elements marked by the dotted lines illustrate their up-and-down or zipped overlapping arrangement. The zipped arrays in the non-folded structures are shown at the left side of FIGS. 7E and 7F, and the zipped arrays in the folded structures are shown at the right side of FIGS. 7E and 7F. Observing toward the surface 140, each transducing element in FIG. 7E is rectangular with gaps between the elements, while, densely packed L-shaped transducing elements in FIG. 7F can minimize the gaps between the elements.

FIG. 8 shows a block diagram of the ultrasound system for improving needle visualization 1 according to one embodiment of this invention. The ultrasound transducer 10 of this invention may be connected to a processing apparatus 70. The processing apparatus 70 can process electric signals of an ultrasonic transducer, in particular, the echoes of the needle obtained by the first column set 142-1 of transducing elements and the second column set 142-2 of transducing elements. The process apparatus 70 may have artificial intelligence programs, such as neural networks, together with computer vision modules, to process the signals carrying the needle image, so as to reconstruct the trajectory of the needle, and advise the user about how to relatively rotate the transducer and the needle to the “needle in-plane” status. Preferably, a 3D or 2D real-time model may be displayed on a screen to show the trajectory of the needle relative to the transducer.

[Three-Longitudinal-Plane (3L) Linear Ultrasound Transducers]

The ultrasound transducer 20 of this invention has a tail 12 and a head 14. A plurality of transducing elements 142 are embedded in a surface 140 of the head 14. The transducing elements 142 are arranged in a (M+1)×N array, that is, (M+1) columns multiplying N rows, wherein M is a positive even number, N is a positive integer, and N is greater than M. In a “non-folded” transducer, M may be 2, . . . , 8, 16, 24, or 32, for example.

The (M+1)×N array is a modification of the aforementioned M×N array (M columns multiplying N rows) by adding a central column of transducing elements at the central gap, which further minimizes the blind zone while keeping the “incomplete mixing technique of bilateral soundwaves” of the 2L (or even-number-longitudinal-plane) transducer.

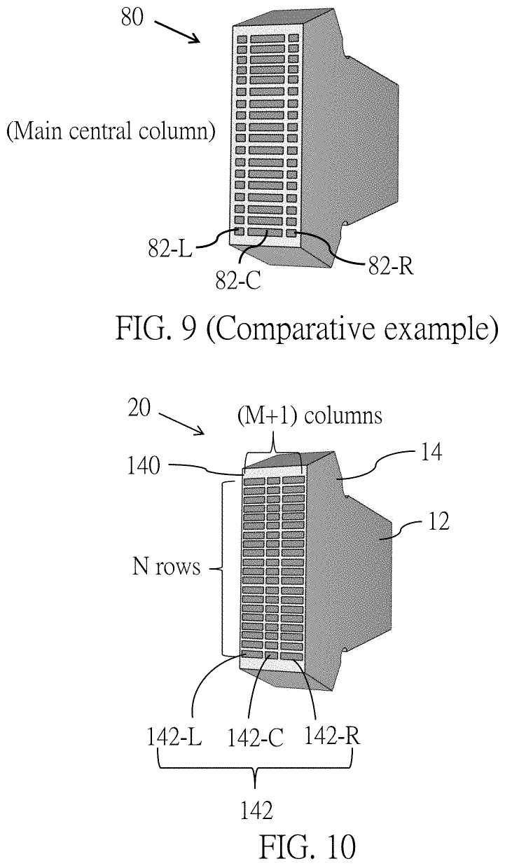

FIG. 9 shows a perspective drawing of the three-longitudinal-plane (3L) linear ultrasound transducer 80 having a main central column according to one comparative example of this invention.

FIG. 10 shows a perspective drawing of the three-longitudinal-plane (3L) linear ultrasound transducer 20 having an ancillary central column according to one embodiment of this invention.

In case of (M+1)=3, the (M+1)×N array is an array having three columns and N rows, and dividing the transducing elements 142 into a left column set of transducing elements 142-L (“left column”, hereinafter), a central column set of transducing elements 142-C (“central column”, hereinafter), and a right column set of transducing elements 142-R (“right column”, hereinafter), configured to form a left ultrasonic longitudinal detection plane PL (namely the “left plane”), a central ultrasonic longitudinal detection plane PC (namely the “central plane”), and a right ultrasonic longitudinal detection plane PR (namely the “right plane”), respectively.

The ultrasound transducer 80 in FIG. 9 is a 3L transducer of a comparative example with a “main central” column design. Notably, the “main central” column design is to emphasize or better visualize the central image plane, by increasing the area or the size of the transducing element in the central column, or by increasing the effective sum area of the whole central column with variable sizes of transducing elements. In FIG. 9, the length of the transducing element of the central column set of transducing elements 82-C is greater than the length of the transducing element of left and right column sets of transducing elements 82-L and 82-R.

In the contrary to FIG. 9, FIG. 10 illustrates the “3L ancillary central” column ultrasound transducer 20 of this invention. In order to further mitigate the blind zone Z while keeping the “incomplete mixing of bilateral soundwaves” of the 2L transducer, an “ancillary central” column of transducing elements 142-C is added in the central gap G. Counterintuitively, elements size (or dimension in transverse axis) of the central column set 142-C is smaller than elements size (or dimension in transverse axis) of the left and right column sets 142-L and 142-R. Instead of focusing and imaging independently among three columns (as shown in FIG. 9), the “ancillary central” column 142-C collaborates with the left and right columns 142-L and 142-R to yield high quality central plane image through the “incomplete mixing technique of bilateral soundwaves” as previously noted in FIG. 3B.

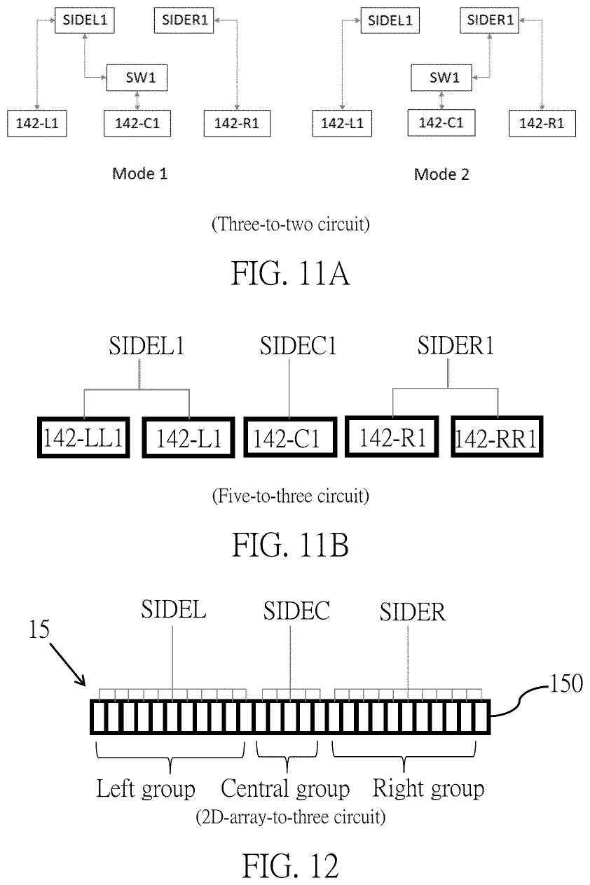

FIG. 11A shows a functional three-to-two circuit block diagram of the three-longitudinal-plane (3L) ultrasound transducer according to one embodiment of this invention.

In FIG. 11A, a three-to-two circuit, which may include a switch (for example, SW1), fulfils signal mixing between the central column (for example, 142-C1) and the bilateral columns set (for example, 142-L1 and 142-R1) of transducing elements in a 3L ultrasound transducer. Through the three-to-two circuit, three array elements in the same row across the three columns (for example, 142-L1, 142C1, 142-R1) are converted and sent to two-channel circuits (for example, SIDEL1 and SIDER1). In this way, the circuitry of the 3L ultrasound transducer may be equivalent to a 2L ultrasound transducer, or the 3L ultrasound may actually be mix designed as “3L at transducing element side, 2L at circuit side”. The mix design of 3L and 2L by the three-to-two circuit has certain advantages. Compared with the “standard 2L” ultrasound transducer, soundwaves in the 3L ultrasound transducer are more effectively transmitted to and received from the central blind zone Z, since only the 3L ultrasound transducer has a central column of transducing elements. Compared with the “standard 3L” ultrasound transducer with a three-channel (in one row) circuits, a 3L ultrasound transducer with a built-in three-to-two circuit has less wiring thickness and weight, and it achieves the “incomplete mixing of bilateral soundwaves” in the transducer.

Specifically, referring to FIG. 11A, in mode 1, the signal from the first-row-left-column transducing element 142-L1 and the signal from the first-row-central-column transducing element 142-C1 are mixed, and processed by a left side circuit SIDEL1. Similarly, in mode 2, the signal from the first-row-central-column transducing element 142-C1 and the signal from the first-row-right-column transducing element 142-R1 are mixed, and processed by a right side circuit SIDER1. It is possible to use a switch SW1 to select which side (e.g. left side, right side, or both side) circuit the signal from the first-row-central-column transducing element 142-C1 is sent to. In this way, the “incomplete mixing of bilateral soundwaves” can be realized in the 3L system, and minimize the blind zone Z locating in the center.

FIG. 11B shows a five-to-three functional circuit block diagram of the five-longitudinal-plane (5L) ultrasound transducer according to another embodiment of this invention.

FIG. 11B extends the principle in the embodiment of FIG. 11A. The signals from five columns of transducing elements 142 may be converted and sent to three side circuits. Specifically, the signal from a first-row-leftmost-column transducing element 142-LL1 and the signal from a first-row-left-column transducing element 142-L1 may be mixed and processed by a left side circuit SIDEL1; the signal from a first-row-central-column transducing element 142-C1 is processed directly by a central circuit SIDEC1; the signal from a first-row-right-column transducing element 142-R1 and the signal from a first-row-rightmost-column transducing element 142-RR1 may be mixed and processed by a right side circuit SIDER1.

The principle is applied analogously to other rows for a specific number of columns. The principle is also applied analogously to other cases with variable numbers of columns. In other words, a plurality of columns of transducing elements may be linked to or processed by a single side circuit; or a signal transducing element may be divided into a plurality of sub-elements, and then recovered by a side circuit.

In series with a three-to-two circuit in FIG. 11A, the five-to-three circuit in FIG. 11B may serially convert a 5L transducer to 3L and then 2L equivalents. Compared with the “standard 5L” ultrasound transducer with a five-channel (in one row) circuits, a 5L ultrasound transducer with a built-in five-to-three and three-to-two circuits has less wiring thickness and weight, and it achieves the “incomplete mixing of bilateral soundwaves” in the transducer.

FIG. 12 shows a 2D-array-to-three functional circuit block diagram of the ultrasound transducer utilizing the 2D-array-type transducing element module 15 according to one embodiment of this invention.

The 2D-array-type transducing element module 15 intrinsically has a plurality of micro transducing elements 150, and they are divided into a plurality of groups. One group of micro transducing elements 150 may be used as a single transducing element 142. In FIG. 12, a plurality of micro transducing elements 150 of the 2D-array-type transducing element module 15 are divided into a left group (which functions as the transducing element 142-L1 in FIG. 11A), a central group (which functions as the transducing element 142-C1 in FIG. 11A), and a right group (which functions as the transducing element 142-R1 in FIG. 11A). The functions of the respective groups in FIG. 12 may refer to the functions of the respective transducing elements in FIG. 11A, so the details are omitted here.

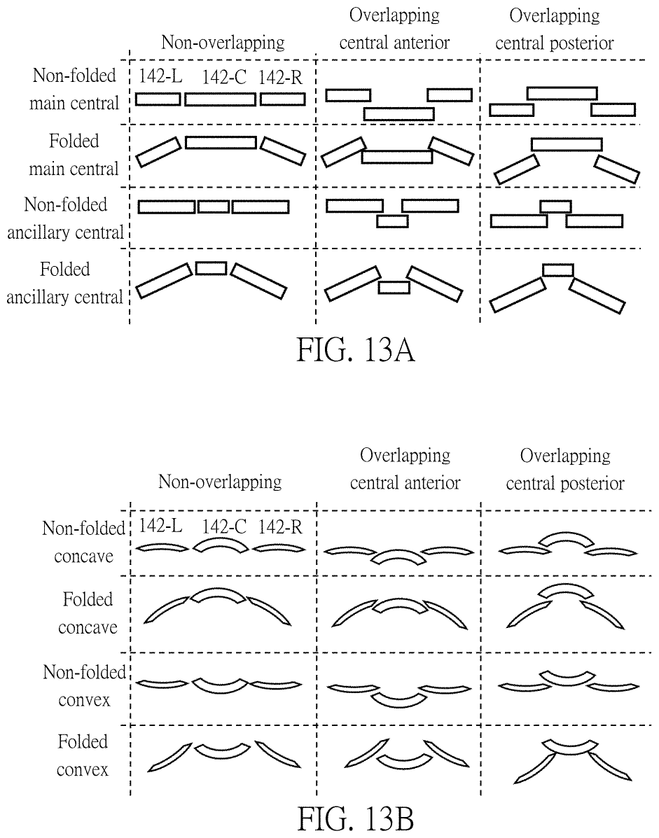

FIGS. 13A and 13B show the configurations of the three-longitudinal-plane (3L) ultrasound transducers according to several embodiments of this invention. Herein, three transducing elements in the left column 142-L, the central column 142-C, and the right column 142-R in one row are schematically shown. To remain clearness of the drawing, the referential numbers 142-L, 142-C, 142-R are labeled only in the upper left diagrams of FIGS. 13A and 13B, the corresponding components in the remaining diagrams of FIGS. 13A and 13B can be identified according to their corresponding positions.

First, in terms of the locations of the transducing elements, three transducing elements in the left column 142-L, the central column 142-C, and the right column 142-R may be arranged non-overlapping, overlapping and “central anterior” (the central column is nearer to the object of interest), or overlapping and “central posterior” (the central column is farther from the object of interest).

Second, in terms of the directions of the transducing elements, the transducing elements may be configured non-folded or folded (which may refer to the folded structure of the 2L transducer in FIG. 6A). Finally, in terms of the sizes (e.g. dimensions in transverse axis of the transducer) of the transducing elements, the “main central” type means that the size of the transducing elements in the central column is greater (or longer) than the size of the transducing elements in the left column or in the right column. The “ancillary central” type means that the size of the transducing elements in the central column is smaller (or shorter) than the size of the transducing elements in the left column or in the right column.

Accordingly, the aforementioned different configurations can have different effects in soundwave overlapping or mixing, and thus provide different depths of image focus and needle trajectory detection. Furthermore, the ultrasound transducer may be designed to allow user to adjust (e.g. move, rotate, or scale up or down) the position, the direction, or the size of the transducing element.

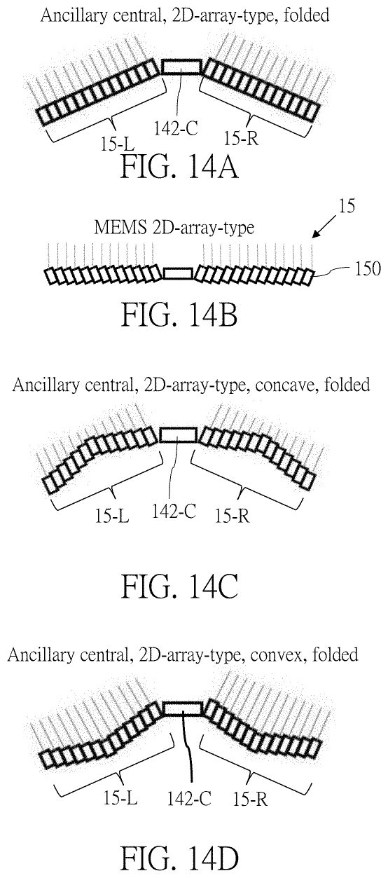

The shape of a single transducing element may be rectangular, as shown in FIG. 13A. Alternatively, it may be bended with a concave surface or a convex surface, as shown in FIG. 13B, which respectively provide the converging effect or diverging effect of the soundwaves for different level of bilateral soundwaves mixing. In FIG. 13B, “concave” means the case where the concave surface faces the object of interest, and “convex” means the case where the convex surface faces the object of interest. FIGS. 14A, 14B, 14C, and 14D show the configurations of the ultrasound transducer utilizing the 2D-array-type transducing element module 15 according to several embodiments of this invention. As an example, the common feature in FIGS. 14A, 14C, and 14D is that the central column transducing element 142-C is farther from the object of interest than the left and right 2D-array-type transducing element modules 15-L and 15-R are. On the other hand, in FIGS. 14A, 14B, 14C, and 14D, the size (or dimension in the transverse axis) of the central column transducing element 142-C is smaller than the size (or dimension in the transverse axis) of the left and right 2D-array-type transducing element modules 15-L and 15-R, which also belongs to the “ancillary central” design as previously described. As shown in FIG. 14B, it is possible to use a microelectromechanical system (MEMS) to form the micro transducing elements 150 of the 2D-array-type transducing element modules. In this case, although the micro transducing elements are arranged in a line, all of them aim at the central plane PC (referring to FIGS. 6C and 10).

As shown respectively in FIGS. 14C and 14D, the left and right 2D-array-type transducing element modules 15-L and 15-R may be bended to a concave surface or a convex surface, to aim at the blind zone or the object of interest, by misaligning the micro transducing elements to each other. Its principle may refer to FIG. 6E and its relevant descriptions, so the details are omitted here. In FIGS. 14C and 14D, “concave” means the case where the concave surface faces the object of interest, and “convex” means the case where the convex surface faces the object of interest.

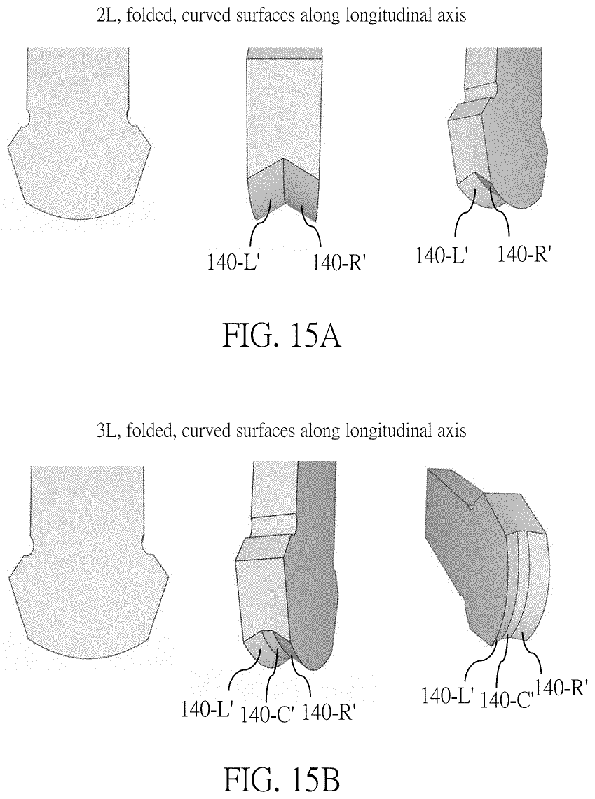

FIGS. 15A and 15B respectively show side views, first perspective drawings, and second perspective drawings of the 2L folded transducer and the 3L folded transducer with curved surfaces along the longitudinal axis.

Comparing with FIG. 2, 6A, or 10, wherein the surface 140, the first regional surface 140-1, and the second regional surface 140-2 are plane (or flat) surfaces. In FIGS. 15A and 15B, all the regional surfaces 140-R′, 140-C′, 140-L′ are curved surfaces along the longitudinal axis of the transducer. Moreover, the regional surfaces 140-R′ and 140-L′ may be non-folded or folded (as depicted here) to each other. Intuitively, the arrangement of the 2×N and 3×N transducing elements of the transducers in FIGS. 15A and 15B, though not depicted, may still refer to FIG. 2, 6A, or 10 and their relevant descriptions.

(It should be noted that, the convex shape or the concave shape of a single transducing element 142 or a 2D-array-type transducing element module 15 refers to curvature along the transverse axis of the transducer. While, the curved shape of surfaces 140-R′, 140-C′, 140-L′ in FIGS. 15A and 15B refers to curvature along the longitudinal axis of the transducer, which should not be confused.) The feature of the curved surface along the longitudinal axis in this invention is exemplified as a transducer having a folded structure with two longitudinal curved surfaces (called “2L curved, folded”) and a transducer having a folded structure with three longitudinal curved surfaces (called “3L curved, folded”). It is to be understood that, the feature of the curved surface in this embodiment may also be applied to the transducers in other embodiments of this invention.

Although the present invention has been explained in relation to its several embodiments, many other possible modifications and variations can be made without departing from the spirit and scope of the invention as hereinafter claimed.

What is claimed is:

1. An ultrasound system for improving needle visualization, comprising an ultrasound transducer, which has a tail and a head; a plurality of transducing elements being embedded in a surface of the head, and the transducing elements are arranged in an array of M columns multiplying N rows, that is, M×N, wherein M is a positive even number, N is a positive integer, and N is greater than M.

2. The system of claim 1, further comprising a central gap on the surface, wherein the central gap divides the array by column equally into a left column set of transducing elements and a right column set of transducing elements, configured to form a left ultrasonic longitudinal detection plane, namely a left plane, and a right ultrasonic longitudinal detection plane, namely a right plane, respectively; wherein there is a blind zone to be minimized under the central gap, or there is a bilateral-equal-time-distance central ultrasonic longitudinal detection plane, namely a central plane, to be built between the left plane and the right plane.

3. The system of claim 2, wherein the central plane is reconstructed by algorithms analyzing time domain signal intensity from the left column set of transducing elements and the right column set of transducing elements, to intensify signal(s) converted from soundwave(s) from the central plane, or to cancel signal(s) converted from soundwave(s) from a position deviated from the central plane.

4. The system of claim 2, wherein the left plane and the right plane are partially mixed based on an incomplete mixing technique of bilateral soundwaves, such that their soundwaves reach a certain level of mixing to build the central plane by signal processing, but at the same time, remain certain levels of their independencies to detect the trajectory of the needle.

5. The system of claim of claim 2, wherein, to mix bilateral soundwaves, the surface of the head is folded to form a left regional surface embedded with the left column set of transducing elements, and a right regional surface embedded with the right column set of transducing elements, the left regional surface is not parallel to the right regional surface; or the surface of the head has a portion being a curved surface.

6. The system of claim 2, wherein each column set of transducing elements itself has a bended structure with a convex surface or a concave surface, aiming at the blind zone or an object of interest.

7. The system of claim 2, wherein, to minimize the blind zone, an acoustic lens is attached in front of the transducing elements; the acoustic lens is configured such that the left plane intersects or partially overlaps the right plane, wherein the acoustic lens is a monofocal lens, or a multifocal lens which has a plurality of focuses; or the acoustic lens has one or more waveguide structures, aiming at the blind zone; wherein the focuses of the acoustic lens locate in line with the central gap, or locate in the central plane or the blind zone; or, the acoustic lens is in mirror symmetry with respect to the central plane.

8. The system of claim 7, wherein, to minimize the blind zone, a splitter is arranged between the transducing elements and the acoustic lens, or inside the acoustic lens, or in front of the acoustic lens, and the splitter aims at the blind zone; or, a splitting structure is formed in the acoustic lens, and the splitting structure aims at the blind zone.

9. The system of claim 2, wherein the array is a staggered array, such that the left column set of transducing elements is misaligned from the right column set of transducing elements; or the array is a zipped array.

10. The system of claim 2, further comprising an auxiliary signal source, attached on the ultrasound transducer or embedded inside the ultrasound transducer; the auxiliary signal source including one or more inertial measurement units, metal detectors, or magnetic sensors.

11. An ultrasound system for improving needle visualization, comprising an ultrasound transducer, which has a tail and a head; wherein the head of the ultrasound transducer has surfaces embedded with a left 2D-array transducing elements module and a right 2D-array transducing elements module; wherein the modules are folded to each other, or not parallel to each other; or each module is composed of mutually misaligned micro transducing elements spreading on a convex, a concave, or a plane surface at the head, such that the modules aim at a blind zone or an object of interest.

12. The system of claim 2, wherein, to decrease the size of the central blind zone, the central gap is minimized; or a central column of transducing elements is inserted at the central gap, and extends the array of M×N into an extended array of (M+1)×N, wherein the extended array is divided into a left column set of transducing elements and a right column set of transducing elements by the central column of transducing elements, configured to form the left plane, the right plane, and the central plane, respectively.

13. The system of claim 12, wherein the central plane is built by algorithms analyzing the time domain signal intensity from the left column set of transducing elements, the central column of transducing elements, and the right column set of transducing elements, to intensify the signal(s) converted from the soundwave(s) from the central plane, or to cancel the signal(s) converted from the soundwave(s) from a position deviated from the central plane; thus, for a minimal array of 3×N when M equals two, a bilateral-augmented central plane is built.

14. The system of claim 12, wherein, to partially mix the bilateral soundwaves, an acoustic lens is attached in front of the transducing elements, such that the left plane intersects the right plane at the central plane.

15. The system of claim 12, wherein, to partially mix the bilateral soundwaves, signal(s) from the left column set of transducing elements and signal(s) from the central column of transducing elements are mixed and processed by a left side circuit; the signal(s) from the central column of transducing elements and signal(s) from the right column set of transducing elements are mixed and processed by a right side circuit.

16. The system of claim 12, wherein the left column set of transducing elements and the right column set of transducing elements have equal transverse dimension, and their transverse dimensions are equal to or wider than transverse dimension of the central column of transducing elements.

17. The system of claim 12, wherein a left transducing element and a right transducing element have equal transverse dimension, and their transverse dimensions are equal to or wider than transverse dimension of a central transducing element.

18. The system of claim 12, wherein the central column of transducing elements is not parallel to the left column set of transducing elements, and/or is not parallel to the right column set of transducing elements.

19. The system of claim 12, wherein the central column of transducing elements is nearer to an object of interest than the left column set of transducing elements is, and/or than the right column set of transducing elements is; or the central column of transducing elements is farther away from an object of interest than the left column set of transducing elements is, and/or than the right column set of transducing elements is.

20. The system of claim 12, wherein at least one of the transducing elements has a bended structure with a convex surface or a concave surface, aiming at a blind zone or an object of interest.
